(19) United States
(12) Patent Application Publication
(10) Pub. No.: US 2022/0008493 A1
(43) Pub. Date: Jan. 13, 2022

(54) METHOD FOR INCREASING THC/CBD CONTENT IN A CANNABIS PRODUCT

(71) Applicant: Byrn Brands, LLC, Miami, FL (US)

(72) Inventors: Danen Thibodeau, Osprey, FL (US); Bill Plifka, Sarasota, FL (US)

(73) Assignee: Byrn Brands, LLC, Miami, FL (US)

(21) Appl. No.: 17/485,446

(22) Filed: Sep. 26, 2021

Related U.S. Application Data
(63) Continuation-in-part of application No. 15/694,220, filed on Sep. 1, 2017.

(51) Int. Cl. A61K 36/185; A61K 31/05; A61K 31/352; G06Q 30/02

(52) U.S. Cl. CPC A61K 36/185; A61K 31/05; A61K 2236/37; G06Q 30/0283; A61K 2236/15; A61K 31/352

(57) ABSTRACT

A method for increasing THC or CBD content, or both, in a cannabis product.

A profile is selected with at least two components, one of which is a wet extract, and combining them into a combined cannabis product. The combined cannabis product is freeze-dried, resulting in at least one wet extract component drawing inward and away from the surface.

A second portion of at least one component containing THC or CBD, or both can then be added to the surface, enhancing the amount of THC and/or CBD content of the product.

### CROSS REFERENCE TO RELATED APPLICATION(S)

This application is a Continuation in Part of, and claims the benefit of, U.S. Utility application Ser. No. 15/694,220 entitled “PHARMACEUTICAL COMPOSITION CONTAINING VARIOUS FORMS & STRAINS OF CANNABIS AND PROCESS FOR FORMING SAID COMPOSITION,” filed on Sep. 1, 2017. The subject matter of this application is hereby incorporated by reference in its entirety.

### TECHNICAL FIELD OF THE INVENTION

The present invention relates to the general art of Cannabis and creation of targeted Cannabis blends, and more specifically to a system and method for calculating and creating targeted blends.

### BACKGROUND OF THE INVENTION

Cannabis is a species of flowering herb that produces flowers (i.e., buds) that are harvested, dried, and used. For literally thousands of years, various peoples have recognized the beneficial effects of Cannabis, using it for spiritual, religious, recreational, and medicinal purposes. There are generally three subspecies of Cannabis: sativa, indica, and ruderalis. (At this time, attention and effort are typically focused on the sativa and indica subspecies, such that the three groupings for use tend to be sativa, indicia, and hybrids.) Within these subspecies, there are a variety of Cannabis plants, from which a large variety of different strains, possibly in the thousands, can be bred. From these, a multitude of combinations can be combined and created.

Cannabis plants are comprised of hundreds of compounds with cannabinoids (i.e., compounds derived from Cannabis) being of great importance. Foremost among these cannabinoids are THC (i.e., tetrahydrocannabinol) and CBD (i.e., cannabidiol). THC can bring on a state of euphoria commonly known as being “high,” as well as provide relief for a number of conditions such as anxiety, depression, and insomnia. CBD is non-intoxicating and is known to help alleviate pain, inflammation, and a number of ailments.

Terpenes are another important compound. Terpenes are aromatic compounds commonly produced by a number of plants that provide odors and flavors. They can be found in Cannabis as well as, for example, oranges, tomatoes, and flowers. Over 20,000 terpenes have been identified overall in plants. Cannabis alone appears to have at least 100, with an exact number not yet clear. In Cannabis, terpenes can give a wide range of smells, such as that of berries or pine. The types and amounts of terpenes in a Cannabis strain can affect the feeling and quality of the using experience and may influence the effects of different Cannabis strains.

Generally, it is thought in the field that there is a primary difference between the effects on mind state and energy level of a user, depending on the plant strain.

Saliva strains are believed to enhance energy and mental activity such as creativity. Indica strains, however, are generally thought to decrease energy and increase relaxation. This perception is so widespread a common nickname for indica is “In Da Couch.”

Cannabis has a number of strains within it; for example, tomatoes or grapes have a number of variations. In recent years, growers have begun to focus on growing strains within both subspecies to create suitable strains for a number of purposes, such as relaxation, increase in focus, or alleviation of pain or other medical conditions. Users have a wide variety of specific needs, such that a number of specific strains are designed to address these needs. For example, a patient suffering from anxiety does not necessarily want or need a euphoric state, and a patient wanting to relax likely does not want their focus increased at that time. This can be more serious when treating conditions. Sometimes, marijuana is a good, effective, non-addictive alternative to treating certain conditions. Such conditions can include, for example, cancer pain, glaucoma, IBS, PTSD, anxiety, Crohn's disease, nausea, and arthritis.

However, patients suffering from these conditions often have families, jobs, and other responsibilities. They need treatments that are both as effective as possible without other effects that affect other aspects of their lives. The current approach to supplying the best possible treatments for specific needs is, though, somewhat scattershot and random.

It is generally considered and thought that sativa is mentally uplifting and best for treating psychological issues such as anxiety or PTSD, and that indica is physically relaxing and best for physical ailments, particularly pain. Often, dispensaries or other Cannabis sources will point customers generally to sativa for one set of issues and indica for another basically towards what they perceive to be high THC or high CBD content products. This is a very limited and unprecise approach. The terms sativa and indica are separated as strains, not by any distinct effects. A sativa strain is not necessarily always the best choice for energy or alertness, and an indica may not always be the best choice for relaxation.

However, the effect a strain will have upon a user is determined by the type and amount of chemical compounds in the strain. These compounds include THC, CBD, and terpenes. This is also known as “the profile” of a strain. The profile of a strain may or may not coincide with its general reputation. The profiles can vary widely within sativa, indica, or hybrid strains.

As one example, a sativa strain known as “Sour Diesel” can have around 20% THC and less than 1% CBD. On the other hand, another sativa strain known as “Charlotte's Web” is nearly reversed; it can have less than 1% THC and around 15-20% CBD. Further, as there are a wide variety of strains and profiles, specific profiles will be more or most effective for specific needs or treatment of specific ailments. Further, there is some evidence that terpenes provide a good deal of, or are at least partly responsible for, specific effects of Cannabis. A theory, undergoing research, is that terpenes and cannabinoids work together synergistically to intensify specific effects, and that terpenes buffer or enhance the effects of the cannabinoids. This is known as “The Entourage Effect.”

Given the number of compounds and complexity of strains deriving from them, recommending a general subspecies for a specific treatment is applying a blunt instrument, which can often be ineffective or even counterproductive. While a number of strains have been bred, particularly hybrid strains, to attempt to enhance or reduce certain effects, the approach of application is still somewhat hit or miss, often depending upon random or semi-random combinations and anecdotal word of mouth. Patients are often left to seek such anecdotal advice, and in the end, rely on a general guess for which strain to use.

Another issue in treating many ailments is that current products for them are costly, difficult to use correctly, often do not effectively address the problem, and some can be addictive. If the product is a narcotic, a patient is supposed to take the narcotics only temporarily because of the damage it can do to the body and its potential to become mentally and physically addictive. Accordingly, narcotics are generally considered to be a short-term treatment that can mask symptoms only.

### SUMMARY

Herein disclosed is a system for creating strains and profiles for strains of Cannabis and effectively targeting strains of Cannabis for specific uses and circumstances.

A strain of Cannabis is generally comprised of differing amounts of varying compounds, which can be broken down and understood further as a specific chemical profile.

Cannabis profiles are comprised of various levels and combinations of cannabinoids and terpenes. Each strain profile has a specific amount and type of chemicals, including CBD, THC and terpenes. In some embodiments, each of these strain profiles is from a different portion of a different type of Cannabis plant.

The method of calibration herein can be used in conjunction with a process for forming targeted Cannabis compositions with unique profiles from Cannabis products. In some embodiments, these can include Cannabis flower from Cannabis leaves and buds (which are typically ground to powder or near powder), oils from the plant, and kief from Cannabis resin glands. However, these can include additional products known in the art, such as, e.g., wet extracts, dry extracts, or a combination of both. These products can have their own unique profiles, which can also be programmed and added into the system. In some embodiments, individual strains can be derived from a different portion of different plants.

In one embodiment, a strain is derived from Cannabis flower from ground leaves, buds, or both. Another strain is derived from Cannabis oil. Yet another strain is derived from Cannabis kief. These have differing formulations, including differing levels of CBD, THC, and various terpenes. As can be seen, each of these representative strains have an individual profile of type and number of active components. These profile strains can be combined into a new profile blend.

In a representative embodiment, the flower from one Cannabis strain, a specific amount of oil from a second Cannabis strain, and a specific amount of kief from a third Cannabis blend, can be combined to create a specific blend.

In alternative embodiments, more than one component of the specific combined blend can come from a single strain, depending upon what the method herein shows to be the best mix. A composition might have flower and oil from a first strain and kief from a second strain, depending upon what is shown to be the best possible combination of strain profiles.

In an embodiment, the ground Cannabis flower and the Cannabis oil, whatever their sources, are combined to produce a mash compound. The Cannabis oil can be warmed, then mixed with the flower for improved combining. The mash compound can also be warmed, for example in an oven, to further liquify any wet extracts, such as oil, to help achieve an even saturation of the wet extract throughout the mash compound.

This mixed mash compound can then be coated with kief in an outer layer. In further embodiments, tetrahydrocannabinolic acid (i.e., THCA, such as THCA in crystal form) or shatter can be substituted for the kief, or used with it as an additional profiled component of the specific blend. The resultant substance can then be separated into smaller individual doses, which can then be distributed and used.

A set of embodiments of the system and method of profile, and of determining which strains of which component, and in what amounts relative to each other to combine, is shown. The method of this embodiment, overall, is to compute and compile possible blends, select target criteria for choosing a blend, filter the target criteria against the database, sort and compare the results, and produce an optimal blend.

In a first and continuing step, a database of strain profiles is provided. Further an algorithm or set of algorithms for processing the various data inputs, plus recommending appropriate strain profiles under various circumstances, is provided and programmed into the system.

The available data about strain profiles is created and loaded into the at least one electronic device to form a database. After creating this database, it is typically routinely updated, with further data added to it.

In addition to strains derived from flower, oil, and kief as mentioned, further unique profiles can be derived from other plant products previously mentioned, such as distillate, flower, rosin, shatter, and sauce. These forms of products and their unique profiles are another factor that can be programmed into the system as additional profiles, and combinations of profiles, for analysis and comparison.

Further, terpenes from other sources can also be profiled and added to any strain profile to achieve such things as new flavors, results, or entourage effects.

To add to an established database or provide as part of setting up a database, the overall blends of compounds are computed by the at least one electronic device.

In a first general step towards this, multiple, variable, available strains and formats are calculated to establish a defined universe of possible strains. The number of possible strains can potentially reach astronomical numbers. Accordingly, parameters are set to establish the universe of possible products to those can be made, considering factors such as type of plants, products, and strains, product inventory, cost effectiveness of strains, availability, the effects of strains on-hand, marketing needs, and profitability.

The mixed ratios of mash, or other Cannabis components, are calibrated to a selection of compounds based on these or other pre-selected factors. Generally, the type of product (e.g., flower, mash, oil) and form (e.g., as smoke-able, edible, etc.) are selected and listed as well. Accordingly, a calculation of the universe of these multiple, variable strains is completed.

In a next general step, the possible permutations of strains in the calculated universe of strains are iterated. Millions of blend permutations may be possible from a compound's selected strain combinations. The specific makeup of each possible profile's contents is quantified according to a pre-determined range of selected ratios of components, such as, e.g., flower to oil to kief. Overall THC and CBD blend values are calculated and listed for each in-universe profile. The entourage value of each blend profile is also computed based upon the profile's unique cannabinoids and terpenes, overall cannabinoid and terpene content, cannabinoid or terpene interactions, and other factors. A plant product's cannabinoid (THC, CBD, etc.) and terpene values are listed and referenced in the database of known strains, including each distinct permutation of these mixtures, to be included in possible blend profiles. From here, the system creates a database of possible profiles, along with the known characteristics of each profile such as, but not limited to, cost, profitability, specific effects, taste, and entourage effects.

After calculating and iterating each unique profile, blend data is formatted for consistency and added to the database for future processing and quick comparisons across multiple iterated profiles within the database.

In a next general step, a set of target criteria is selected by the at least one electronic device.

A user inputs the effects sought and other factors concerning an optimum mix of strain profiles into a specific treatment profile. A creator may select for potency of specific cannabinoids, such as THC or CBD. Or, a creator might opt for the “full spectrum” entourage effect, where the whole is greater than the sum of the parts. A creator can choose profile selections that highlight specific flavors and aromatics, such as bright, citrusy flavors or earthy or piney undertones. A creator can also choose to decrease costs and/or increase profits by including strains and plant products based on current inventory availability and market value.

In another step, several factors are filtered against the known strain profile information and choices, in the database by the at least one electronic device. In one embodiment, the at least one electronic device completes the process via a pre-programmed algorithm, or one of a set of algorithms. The selected targeted criteria is/are used for comparison with provided blend profiles in the database, compared against the profiles, and filtered against known database values for the profiles.

The relative cannabinoid content of each strain is factored and run against the database. For a few examples, when a specific THC/CBD potency or other entourage effect is selected, the system can query the database, and the optimized profile from all possible combinations can quickly be retrieved and sorted from database records based on pre-calculated profile values.

The relative terpene content, including types of terpenes and amounts in each strain, is factored and run against the database. For example, when a specific taste is selected, the system can query the database to retrieve known terpene values and select the optimum stored blend by weighing a profile's distinct flavors, aromatics, and terpene interactions. The relative flavor of each terpene, and level of flavor in each strain, can be factored and run against the database. Terpene interactions, with each other, and with the cannabinoids in each strain, can also be factored and run against the database.

A specific treatment option can be selected. For a few examples, a treatment option could be, e.g., treating a specific ailment, treating symptoms, or even simply providing an optimum recreational experienced. When a treatment option, such as, e.g., targeting a specific aliment, is selected, terpene and cannabinoid interactions and effects are retrieved from the database specific to that treatment option. The specific ailments to be treated, or other effect sought, and the best-known compounds, or strain profiles, for each can be factored and run against the database. The at least one electronic device is used to select a blend by the system based on the strongest overall effect possible from stored database profiles, thereby selecting the strongest treatment option.

When cost-centric criteria are selected as the target criteria, the database is consulted for product inventory levels, costs of production, market popularity, and retail values to assign a currency value per unit per blend and select the optimized cost profile. Further, market popularity of each strain may also be factored and run against the database.

In addition, in other embodiments, these and additional factors can be entered together and given weighted values, resulting in an overall profile that provides a balance of multiple factors.

In another overall step, the results of the previous steps are sorted and compared by the at least one electronic device.

As part of this, the system sorts the various aggregate values (e.g., treatment sought, cost, availability of specific strains, entourage effect, etc.) that are programmed into the system and sorts and compares them relative to each other. In one embodiment, this process is completed by the algorithm, or one of a set of algorithms.

Based upon the results of this sorting, the system selects a maximized target blend. After a calibration is complete, the results can provide a formulation for combining ingredients from different Cannabis plants, such as flowers, oils, and/or kief.

From here, an optimum blend is produced in a next overall step.

As part of this overall step, a specific calculated blend is represented in some form so that it can be produced in one embodiment, an optimized data set for a specific blend is graphed and a dynamic graph is presented for customizing the blend. The graph can be a visualization tool for the creator showing the individual plant product and blend aggregate content values of the selected compound, representing cannabinoids, terpenes, strains, or effects. It can also be a set of internal programmed instructions for the at least one electronic device to carry out, or a mix of both.

Further, a blend can be the result of pre-programmed instructions to the system, or the blend creator may choose to interactively customize a blend profile not optimized for a target criteria (i.e., best THC/CBD, best entourage effect, etc.) via an interface and view the results.

The calibration process provides a methodology of combining these components into compositions for targeted purposes. The system can provide effective, targeted, and accurate combinations and dosages of effective ingredients for any of multiple purposes, including, but not limited to, medicinal, ailment relief, pain and inflammation relief, recreational, or psychological help. With the additional factors and possible combinations of compounds that can be processed and used, the system can increase the potential number of available strains beyond the current limited number of possibilities into an additional realm of possible combinations and profiles.

Further, additional steps and factors can be incorporated into the system and method herein to provide a number of additional benefits. Currently known entourage effects can be programmed into the database to include and factor these is as well. In addition to providing the most effective combinations for a given need, the system also allows for programming of additional factors that can maximize profits and reduce waste of product, such as maximizing use of product in stock to reduce wastage and lower cost strains when possible. Further, the system can also be programmed to avoid negative consequences, as they become known and entered into the database, such as, for example, counterproductive Cannabis single strains or combinations. Further, tailor-made flavor profiles can be created to provide users with specific flavors and odors to enhance their product experience. These can all lead to increased profitability and increased consumer satisfaction.

The strain profiles can be represented by the system in any way known in the art for relative comparison to each other.

In one embodiment, a “wheel” type representation displays the THC and CBD breakdown of a profile, as well as the layers of product in the profile. In this embodiment, the layered products are oil and flower (combined into a mash) with a coating of kief. In this embodiment, each of the represented product layers is comprised of a single strain.

Further, each product strain layer can be further comprised of multiple strains, in which each circular product layer is comprised of multiple strains, which can be represented by dividing each circular layer into segments. A product can further be created from multiple sub-products, and accordingly, be represented by additional multiple product layers.

In further embodiments, each compound, such as terpene, or strain, can be assigned an individual color for ease of identification. Further, compounds that work on the same or similar purposes can be assigned similar colors.

In other embodiments, the system can be successfully used to either engineer or reverse engineer strains. A set of known profiles can be combined to create products with known, or likely, results. Products can also be reverse engineered, with a creator knowing and entering the results desired, and the database working backward from there to provide a profile most likely to deliver these results.

Other embodiments are drawn to a process that involves blending and infusing a Cannabis product, which in some preferred embodiments, is a combination of Cannabis wet extracts, or wet and thy extracts, into a mash product, or other product, and then freeze drying the Cannabis mash product.

In a preferred embodiment, a method is shown that is drawn to a double blending process of the specific terpene and cannabinoid profiles created by the system and method herein.

In a first step, the amount of THC and/or CBD percentage(s) desired are entered into the system as parameters, using the electronic device.

In a next step, the system processes the entered (and possible pre-entered) parameters and delivers a set of profiles that meet high-THC, or high-CBD criteria.

Next, a specific profile of strain(s) and combined components is selected for a resultant profile of component(s).

In a further step, a first portion of the components are mixed and combined together. As steps toward this general step, any flower or coarse material is pound into a fine or medium coarse consistency. In one embodiment, the oil rosin, wet extract, or other liquid or partly-liquid substance is added and warmed, with a heated mixing machine.

In a next step, any rosin, oil, or wet extract, or other substance in liquid or partly-liquid form in the formulation is mixed with any ground flower in the formulation, along with any kief, thca or dry extract.

In other embodiments, some components of a specific profile may be warmed while others are submitted to a cold chain process, and yet others may receive neither treatment, depending upon such factors as THC or CBD level sought, and terpenes to be present.

In a next step, the product which has been combined can be placed on trays and freeze-dried. When the first portion of combined product is freeze dried, the high-THC/CBD components tend to pull away from the surface and saturate inward toward the center through absorption. This has the effect of physically leaving less high-THC/CBD product at or near the product surface.

In a further step, a second portion of THC or CBD containing component or components, of the selected formulation, including components such as oil, rosin, kid, wet, or dry extract, or combination of these from the selected formulation, are heated and combined.

In another step, the heated and mixed second portion is added to the first freeze dried portion, and the two portions combined to form a THC-enhanced or CBD enhanced, or both, formulation.

### BRIEF DESCRIPTION OF THE DRAWINGS

FIG. 1 is a schematic diagram illustrating an aspect of the invention.

FIG. 2 is a schematic diagram illustrating a further aspect of the invention, including that of FIG. 1.

FIG. 3 is a schematic diagram of an embodiment of a method of using the inventive system herein.

FIG. 4 is a schematic diagram of a form of display of a portion of the invention of the embodiment of FIG. 3.

FIG. 5 is another schematic diagram of a form of display of a portion of the invention of the embodiment of FIG. 3.

FIG. 6 is another schematic diagram of a form of display of a portion of the invention of the embodiment of FIG. 3, showing further product layers.

FIG. 7 is a schematic diagram of a portion of an interface to facilitate use of the system herein.

FIG. 8 is a schematic diagram of a portion of an interface to facilitate use of the system herein.

FIG. 9 is a schematic diagram of a portion of an interface to facilitate use of the system herein.

FIG. 10 is a schematic diagram of a representation of a product blend product derived from the system and method herein.

FIG. 11 is a schematic diagram of a more complete visual representation of the blended strain product derived from the method and system herein.

FIG. 12 is a schematic diagram of an alternate visual representation of the product of FIG. 11.

FIG. 13 is a schematic diagram illustrating steps of one embodiment of the invention.

FIG. 14 is a schematic diagram illustrating a particular aspect of the invention.

FIG. 15 is a schematic further illustrating the aspect of the invention of FIG. 14

FIG. 16 is a schematic further illustrating the aspect of the invention of FIG. 14

### DETAILED DESCRIPTION OF PREFERRED EMBODIMENTS

Disclosed herein is a system for creating strains and profiles for strains of Cannabis, and effectively targeting strains of Cannabis for specific uses and circumstances.

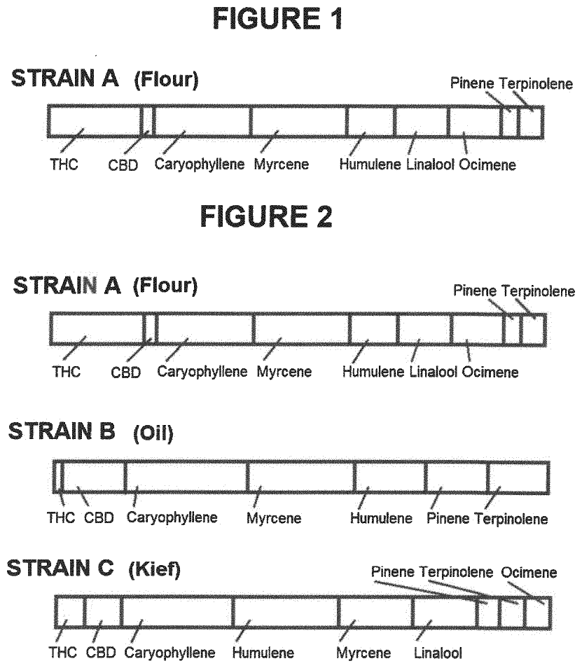

Turning to FIG. 1, an example of a Cannabis profile is shown. For representative purposes, this will be titled “Strain A.”

Each of these representative strains herein, as with strains generally, are comprised of differing amounts of varying compounds, which can be broken down and understood further as a specific chemical profile of the strain, which can be displayed in a number of forms.

Cannabis profiles in the system herein tend to focus on the various levels and combinations of cannabinoids and terpenes. Further, each distinct portion of a Cannabis plant can have a differing profile from the profile of other portions of the same strain. Each of these profiles of a strain has a specific amount and type(s) of chemicals, including CBD, THC and terpenes. Each of these strain profiles in this embodiment, accordingly, is from a different portion of a different type of Cannabis plant. Given that there are over 700 strains of Cannabis, this number is constantly increasing through breeding and hybridization, and each portion of a Cannabis plant can have a different combination of types and amounts of components; there are literally thousands, or more, possible combinations of strains that can be comprised.

This method of calibration herein can be used in conjunction with a process for forming targeted Cannabis compositions with unique profiles from Cannabis products. In the next few representative examples, these will include:

Cannabis flower from Cannabis leaves and buds (which are typically ground to powder or near powder), oils from the plant, and kief from Cannabis resin glands.

Additional products known in the art, such as, e.g., Cannabis dry extracts, such as, but not limited to, kief, THCA, CBDA, hash, or the like, as well as, e.g., Cannabis wet extracts that are created in various ways using methods which include techniques that require butane, co2, ethanol, alcohol, or water and ice extraction. These methods can produce bubble hash, shatter, crumble, budder, rosin, diamonds, sauce, or distillate.

These products can have their own unique profiles, which can also be programmed and added into the system.

In this embodiment, Strain A is comprised of a high THC content of 17%, a low CBD content of 1%, and a representative mix of terpenes. For purposes of this example, several representative terpenes will be shown, though as stated herein, there over one hundred 100 terpenes in Cannabis.

In this embodiment, a representative limited mix of terpenes is depicted: caryophyllene, myrcene, limonene, humulene, linalool, pinene, terpinolene, and ocimene. In this embodiment, there is a higher amount of caryophyllene and myrcene present, while pinene and terpinolene appear in the lowest amounts.

Turning to FIG. 2, several representative strains—Strain A, Strain B, and Strain C—are shown. For purposes of illustration, these three representative strains are derived from a different portion of three different plants and will be used to illustrate some details about the invention.

The representative Strains A, B, and C are comprised of the following:

Strain A—Is derived from Cannabis flower from ground leaves, buds, or both. The Strain A profile has a high THC content of 17%, a low CBD content of 1%, and a representative mix of terpenes, with a higher amount of caryophyllene and myrcene present, some humulene, linalool, and ocimene, and lesser amounts of pinene and terpinolene.
Strain B—Is derived from Cannabis oil. The Strain B profile has a low THC content of 1%, a high CBD content of 15%, and a representative mix of terpenes, with a higher amount of caryophyllene and myrcene present, and some humulene, pinene and terpinolene.
Strain C—Is derived from Cannabis kief. The Strain C profile has a medium THC content of 8%, a medium. CBD content of 10%, and a representative mix of terpenes, with a higher amount of caryophyllene and humulene present, some, myrcene, linalool, and lesser amounts of pinene, terpinolene, and ocimene.

As can be seen, each of these representative strains can and does have an individual profile of type and number of active components. In an embodiment, each of these profiles, from each of these profiles—Strain A, Strain B, and Strain C—is combined into a new profile blend.

The flower from Cannabis Strain A, a specific amount of oil from Cannabis Strain B, and a specific amount of kief from Cannabis Blend C, can be combined to create a specific blend.

In alternative embodiments, more than one component of the specific combined blend can come from a single strain, depending upon what the method herein shows to be the best mix. For example, a composition might have oil and kief from a Strain A and Strain B, and flower from another strain C. Or a composition might have flower and oil from Strain A, and kief from Strain C, depending upon what is shown to be the best possible combination of strain profiles.

Further, the blend components can be of different or similar physical forms. The components can be, for example, comprised of all dry components, all wet- or oil-based, components, crystalline components, or a combination of these.

In an embodiment, the ground Cannabis flower and the Cannabis oil, whatever their sources, are combined to produce a mash compound. The Cannabis oil can then be combined with the mash. The Cannabis oil can be warmed, then mixed with the flower for improved combining. The mash compound can also be warmed, for example in an oven, to further liquify the oil, to help achieve an even saturation of the oil throughout the mash compound.

This mixed mash compound can then be coated with the kief in an outer layer of the kief. In further embodiments, THCA, such as THCA in crystal form, or shatter can be substituted for the kief or used with it as an additional profiled component of the specific blend. The resultant substance can then be separated into smaller individual doses, which can then be distributed and used.

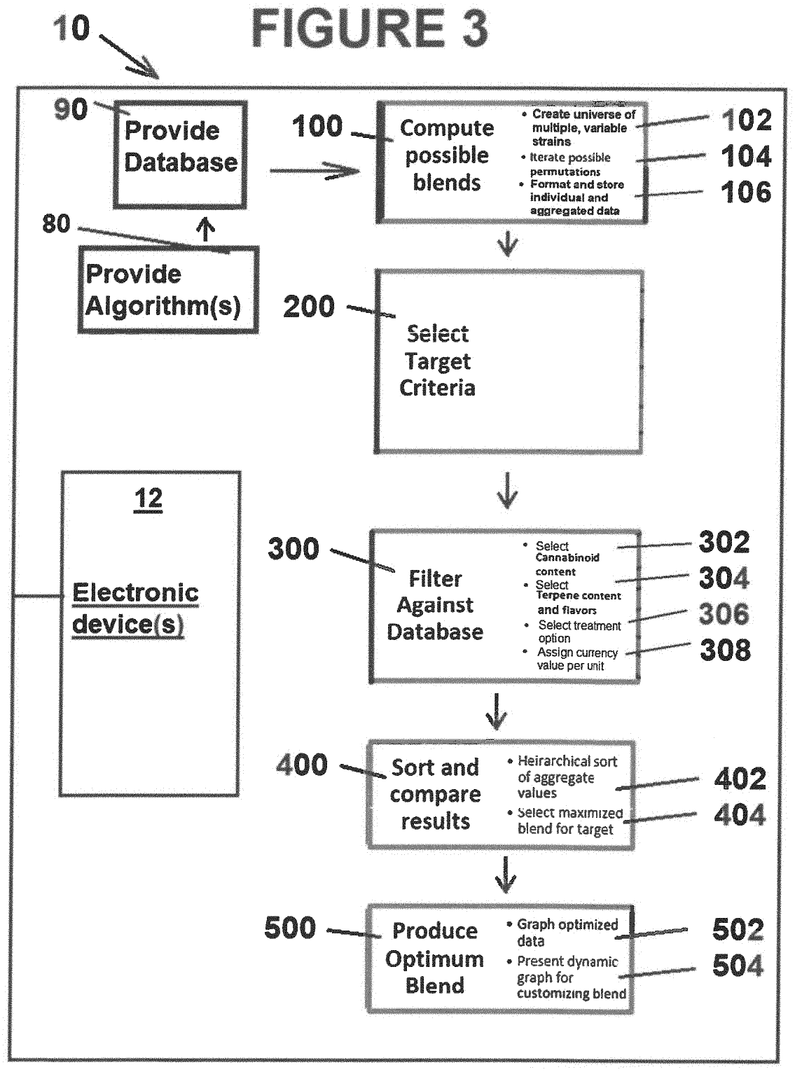

Turning to FIG. 3, a sample embodiment of the system 10 and method of profile, and of determining which strains of which component, and in what amounts relative to each other to combine, is shown and discussed. The method of this embodiment, overall, is to compute and compile possible blends 100, select target criteria 200 for choosing a blend, filter the target criteria against the database 300, sort and compare the results 400, and produce an optimal blend 500.

In a first and continuing step, a database of strain profiles is provided 80. Further an algorithm or set of algorithms for processing the various data inputs, and recommending appropriate strain profiles under various circumstances, is provided and programmed into the system 90. A good deal of information is known about the effects of strains and strain profiles, and combinations of components and relative amounts, on a user. For example, it is known that high levels of THC often create a euphoric feeling in a user. Positive physical effects of CBD are understood to some extent, and some information about the resulting effects of adding specific terpenes and terpene blends is known.

The data about strain profiles is created and loaded into at least one electronic device 12. The at least one electronic device 12 can be any known in the art, such as at least one server, PC, laptop, external drive, tablet, smartphone, or combination thereof. After creating this database, it is typically routinely updated, with further data added to it.

Further research and more information, along with the creation and testing of more strains, will likely result in more thorough and expanding data. For example, some research into the effects of terpenes shows that myrcene may increase appetite. Other terpenes may have properties that have anti-inflammatory, antifungal, and antibacterial properties. There is also ongoing research into the entourage effect between these various components. For example, certain terpenes may work with certain cannabinoids to create or enhance given effects.

In addition to strains derived from flower, oil, and kief as mentioned, further unique profiles can be derived from other plant products previously mentioned, such as distillate, flower, rosin, shatter, and sauce. These forms of products and their unique profiles are another factor that can be programmed into the system as additional profiles, and combinations of profiles, for analysis and comparison.

Further, terpenes from other sources can also be profiled and added to any strain profile to achieve such things as new flavors, results, or entourage effects.

To add to an established database or provide as part of setting up a database, the overall blends of compounds are computed 100 by the at least one electronic device 12.

In a first general step towards this, multiple, variable, available strain and formats are calculated to establish a defined universe of possible strains 102. The number of possible strains can potentially reach astronomical numbers. Accordingly, parameters are set to establish the universe of possible products to those can be made, considering factors such as what plants, products, and strains are available, product inventory, cost effectiveness of strains, availability, the effects of strains on-hand, marketing needs, and profitability. The mixed ratios of mash, or other Cannabis components, are calibrated to a selection of compounds based on these or other pre-selected factors. Generally, the type of product (such as flower, mash, oil) and form (such as smoke-able, edible, etc.) are selected and listed as well. Accordingly, a calculation of the universe of these multiple, variable strains 102 is completed.

In a next general step, the possible permutations of strains in the calculated universe of strains are iterated 104. Millions of blend permutations may be possible from a compound's selected strain combinations. The specific makeup of each possible profile's contents is quantified according to a pre-determined range of selected ratios of components, such as, e.g., flower to oil to kief. Overall THC and CBD blend values are calculated and listed for each in-universe profile. The entourage value of each blend profile is also computed based on the profile's unique cannabinoids and terpenes, overall cannabinoid and terpene content, cannabinoid or terpene interactions, and other factors. A plant product's cannabinoid (THC, CBD, etc.) and terpene values are listed and referenced in the database of known strains, including each distinct permutation of these mixtures, to be included in possible blend profiles. From here, the system creates a database of possible profiles, along with the known characteristics of each profile, such as, but not limited to, cost, profitability, specific effects, taste, and entourage effects 104.

After calculating and iterating each unique profile, blend data is formatted for consistency and added to the database 106 for future processing and quick comparisons across multiple iterated profiles within the database.

In a next general step, a set of target criteria is selected 200 by the at least one electronic device 12.

A user inputs the effects sought and other factors concerning an optimum mix of strain profiles into a specific treatment profile. For example, a creator may want to create a blend that enhances creativity and has a mild physical soothing effect, but without any euphoric or mind hindering effects. A creator may select for potency of specific cannabinoids, such as THC or CBD. Or a creator might opt for the “full spectrum” entourage effect, where the whole is greater than the sum of the parts. A creator can choose profile selections that highlight specific flavors and aromatics, such as bright, citrusy flavors or earthy or piney undertones. A creator can also choose to decrease costs and/or increase profits by including strains and plant products based on current inventory availability and market value 202.

In another step, a several factors are filtered against the known strain profile information and choices, in the database 300 by the at least one electronic device 12. In one embodiment, the at least one electronic device 12 completes the process via a pre-programmed algorithm, or one of a set of algorithms. The selected targeted criteria is/are used for comparison with provided blend profiles in the database, compared against the profiles, and filtered against known database values for the profiles.

The relative cannabinoid content, or a desired range of content, of each strain is factored and run against the database 302. For a few examples, when THC/CBD potency or the “Full Spectrum Entourage Effect” is selected, the system 10 can query the database, and the optimized profile from all possible combinations can be quickly retrieved from sorted database records based on pre-calculated profile values 302.

The relative terpene content, including types of terpenes and amounts in each strain, or a range of content, is factored and run against the database 304. For example, when a specific taste is selected, the system 10 can query the database to retrieve known terpene values and select the optimum stored blend by weighing a profile's distinct flavors, aromatics, and terpene interactions 304. The relative flavor of each terpene, and level of flavor in each strain, can be factored and run against the database. Terpene interactions, with each other and with the cannabinoids in each strain, are also factored and run against the database.

A specific treatment option can be selected 306. For a few examples, a treatment option could be, e.g., treating a specific ailment, treating symptoms, or even simply providing an optimum recreational experience. When a treatment option, such as, e.g., targeting a specific aliment, is selected, terpene and cannabinoid interactions and effects are retrieved from the database specific to that treatment option. The specific ailments to be treated, or other effect sought, and the best-known compounds, or strain profiles, for each can be factored and run against the database. The at least one electronic device 12 is used to select a blend by the system 10 based on the strongest overall effect possible from stored database profiles, thereby selecting the strongest treatment option 306.

When cost-centric criteria are selected as the target criteria, the database is consulted for product inventory levels, costs of production, market popularity, and retail values to assign a currency value per unit per blend and select the optimized cost profile 308. For example, production costs and availability of each strain can also be factored and run against the database.

As an example of this, if Strains B and F are roughly equally effective when combined with other strains in an overall profile, but B is less expensive than F, or E is more readily available in stock, than the parameters can be set within the system in which it will, for cost reasons, select E.

Further, market popularity of each strain may also be factored and run against the database. As an example, if Strain and another strain known as “Blue Turtle” are roughly equally effective, alone or in combination with other strains for treating PTSD, and the Blue Turtle is far more popular and well-known, the system 10 can be programmed to add in the more popular Blue Turtle Strain.

In addition, in other embodiments, these and additional factors can be entered together and given weighted values, resulting in an overall profile that provides a balance of multiple factors. For example, a request to select a profile based 50% on cost and 50% on flavor can be entered, in which case both would be given equal calculated weight in creating a profile that partially or completely meets both criteria.

In another overall step, the results of the previous steps are sorted and compared 400 by the at least one electronic device 12.

As part of this, the system sorts the various aggregate values (e.g., treatment sought, cost, availability of specific strains, entourage effect, etc.) that are programmed into the system and sorts and compares them relative to each other 402. In one embodiment, this process is completed by the algorithm, or one of a set of algorithms.

Based upon the results of this sorting, the system selects a maximized target blend 404. After a calibration is complete, the results can provide a formulation for combining ingredients from different Cannabis plants, such as flowers, oils, and/or kief.

It is noted that in another embodiment, the components such as flower, oil, and/or kief can be from a single strain, but the extraction and blending of the distinct components, though from a single strain, can result in a product with a unique strain profile. For example, the percentages of flower, oils, and/or kief can be altered relative to each other to create a number of unique strain profiles. Because of their effects as combined components, the combination of these components into a distinct products with distinct strain profiles can still result in a distinct entourage effect.

From here, an optimum blend is produced 500 in a next overall step.

As part of this overall step, a specific calculated blend is represented in some form so that it case be produced. This can be done by any means known in the art. For example, in one embodiment, an optimized data set for a specific blend is graphed 502 and a dynamic graph is presented for customizing the blend 504. The graph can be a visualization tool for the creator showing the individual plant product and blend aggregate content values of the selected compound, representing cannabinoids, terpenes, strains, or effects. It can also be a set of internal programmed instructions for the at least one electronic device 12 to carry out, or a mix of both.

Further, a blend can be the result of pre-programmed instructions to the system, or the blend creator may choose to interactively customize a blend profile not optimized for a target criteria (i.e., best THC/CBD, best entourage effect, etc.) via an interface, and view the results. In other words, a blend can be created by a set of pre-programmed instructions, through creator direction after the database is set up and relevant factors entered, or a user can follow the steps herein, manually entering parameters, or a combination of these.

The calibration process provides a methodology of combining these components into compositions for targeted purposes. The system can provide effective, targeted, and accurate combinations and dosages of effective ingredients for any of multiple purposes, including, but not limited to, medicinal, ailment relief, pain and inflammation relief, recreational, or psychological help. With the additional factors and possible combinations of compounds that can be processed and used, the system can increase the potential number of available strains beyond the current limited number of possibilities into an additional realm of possible combinations and profiles.

Further, additional steps and factors can be incorporated into the system and method herein to provide a number of additional benefits. Currently known entourage effects can be programmed into the database to include and factor these is as well, In addition to providing the most effective combinations for a given need, the system also allows for programming of additional factors that can maximize profits and reduce waste of product, such as maximizing use of product in stock to reduce wastage and using lower-cost strains when possible. Further, the system can also be programmed to avoid negative consequences, as they become known and entered into the database, such as, e.g., counterproductive Cannabis single strains or combinations. Further, tailor-made flavor profiles can be created to provide users with specific flavors and odors to enhance their product experience. These can all lead to increased profitability and increased consumer satisfaction.

Further, the product can be made by warming the product as part of a decarboxylating warm chain process, primarily to convert any THCA into THC by heating, which raises product effectiveness. In a preferred embodiment, this infusion process is done using a decarboxylating oven.

Additionally, a cold chain method, of any within the art, can be used in making the product to help preserve any vital terpenes and cannabinoids. Cold chain is a process in which specific or all profile strains are kept at a low temperature, or frozen, to preserve the chemical properties of the components within, particularly of the more fragile terpenes. The strain(s) can be kept at a temperature possibly as cold as—120° F. or less.

In other embodiments, some components of a specific profile may be warmed for decarboxylation, while others are submitted to a cold chain process, and yet others may receive neither treatment, depending upon such factors as THC or CBD level sought and terpenes to be present.

The strain profiles can be represented by the system in any way known in the art for relative comparison to each other.

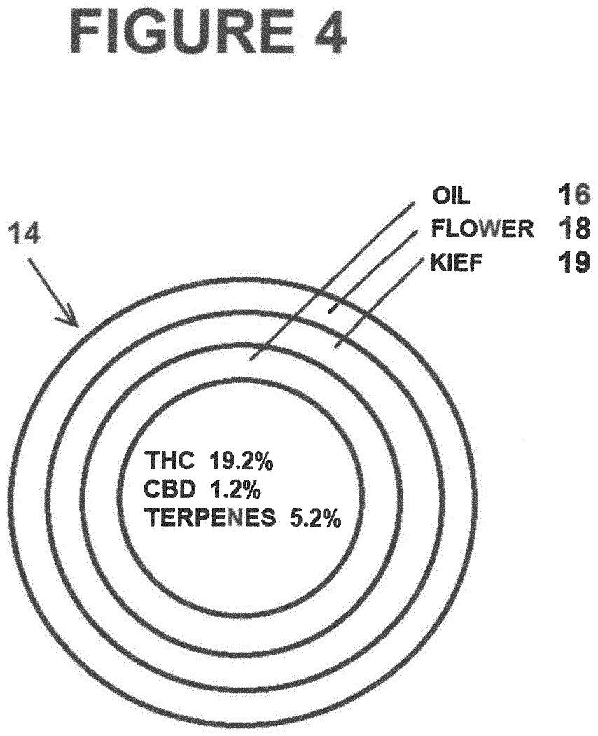

Turning to FIG. 4, a representative strain profile 14 is shown. This “wheel” type representation displays the THC and CBD breakdown of a profile, as well as the layers of product in the profile. In this embodiment, the layered products are oil 16 and flower 18 (combined into a mash) with a coating of kief 19. In this embodiment, each of the represented product layers is comprised of a single strain.

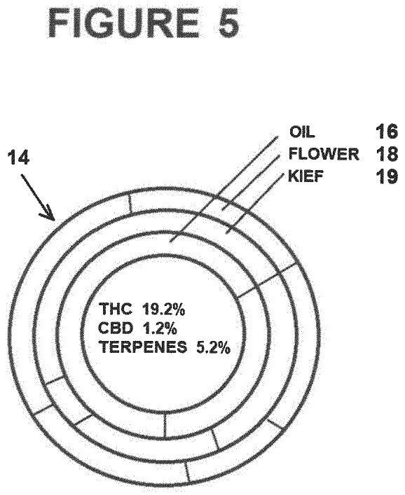

Turning to FIG. 5, each product strain layer can be further comprised of multiple strains. In FIG. 5, each circular product layer comprised of multiple strains, which can be represented by dividing each circular layer accordingly into segments. As shown in this representative figure, the flower component layer of FIG. 5 is shown with five strains, the oil layer with three, and the kief layer with four. As can be seen from the segmented layers of the wheel, each profile layer has differing relative amounts of representative strain components.

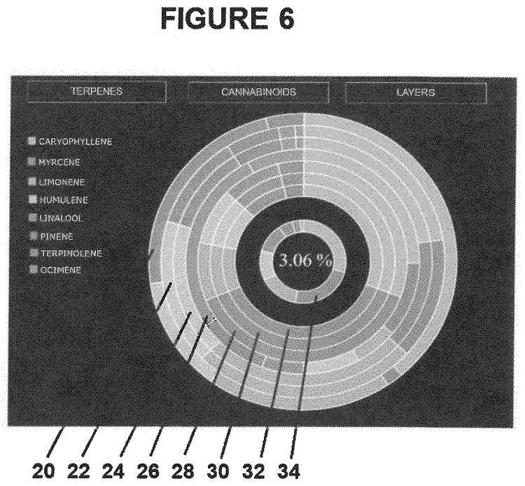

Turning to FIG. 6, a product can further be created from multiple sub-products, and accordingly, be represented by additional multiple product layers. For example, multiple oils or flowers can be used in the same mash, and kief, shatter, butter, or a mix of these added. In this representation, an overall profile comprised of multiple product layer strains, represented herein as 20, 22, 24, 26, 28, 30, 32, 34 is shown. Each segment of each product layer strain can also be color coded differently, to represent multiple strain components to each product.

It is noted that each compound (e.g., terpene) or strain, can be assigned an individual color for ease of identification. Further, compounds that work on the same or similar purposes can be assigned similar colors. As an example, purple could be assigned as a color for anxiety reduction and/or PTSD treatment. Strains or compounds known for effective treatment of such symptoms can be assigned shades of purple, so that when a user or creator sees purple, they can identify the color with these and similar treatments.

The system 10 can be successfully used to either engineer or reverse engineer strains.

A set of known profiles can be combined to create products with known, or likely, results. Products can also be reverse engineered, with a creator knowing and entering the results desired, and the database working backward from there to provide a profile most likely to deliver these results.

### Example 1

A manufacturer wants to create a blend that is optimal for treating the symptoms of PTSD, while also providing a mild anti-inflammatory. Further, the manufacturer has a large amount of Cannabis Strain B that needs to be used, and the user wants to create as low cost a blend as possible. These criteria for a profile are selected and entered into the system 10 to create a set number of possible strains and thereby, derivative mixes of strains, to choose from.

Additional criteria can be entered, including, as here, the condition for treatment, effects sought, and other factors, such as smell or taste of product. The system then determines a profile using the algorithm(s), the provided database, and the at least one electronic device 12. The system 10 also determines a preferred percentage of THC and CBD, respectively, that is needed to formulate a profile, as well as selects a profile of terpene(s) to blend into the product for smell, taste, and entourage effect with other components present in the profile, or other reasons. The system also selects the profile further based on what product, in which percentages, will best provide the sought-after effects. Such products can include the strain of flower, oil, and related products such as kief, rosin, extracts, shatter, or dry or wet Cannabis extract, to complete a specific profile.

Further, in an ongoing process, this profile and its reported results, along with information of other profiles created, used, or known, will be entered into the database to provide a constant updating of the database and cycle of improvement of knowledge and blends.

After the system determines an optimal profile blend based on the various types of input, the components of the product are prepared. If Cannabis flower is to be part of the resultant strain profile, the buds, and possibly leaves, are ground into a very fine flower. If rosin oil, or wet extract, or combination thereof, is to be added, it is placed into a suitable heating machine as known in the art and heated. This decreases the thickness of the oil or similar product, which is then added and mixed with the flower to form a mash. The heating of the oil or similar product allows it to soak and mix more thoroughly into the flower. The resultant mash can be cooled to a suitable temperature, in this embodiment, ambient temperature. The mash can be extruded into pre-selected sizes and shapes and coated with kief, THCA, other suitable product(s).

### Example 2

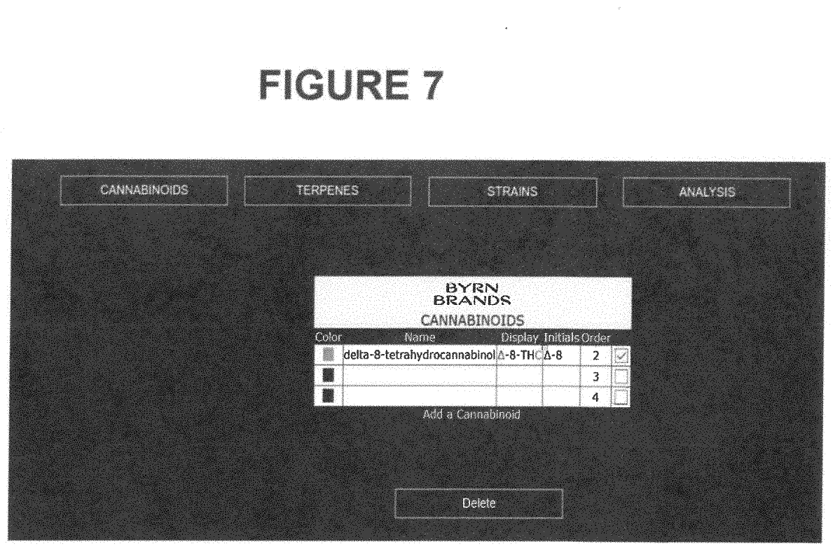

Turning to FIG. 7-12, a representative example of use of the system 10 is shown. Turning to FIG. 7, a portion of an interface for creating a profile—with a visual representation—is shown. In this embodiment, a user can manually enter preferences on strains, terpenes, etc. to create a blend profile. A user can create this at this point, or do so with input received from the system 10 based on previous input of requirements and output provided by the system 10.

Here, a user can first enter preferred cannabinoids. In this embodiment, only one is currently available (though there can be many), so this is the one shown being entered. Buttons are also present for other components, such as terpenes, strains to be added, and analysis to create a blend profile. Also shown in this embodiment, one or more listed products can be deleted if, for example, they are not physically available.

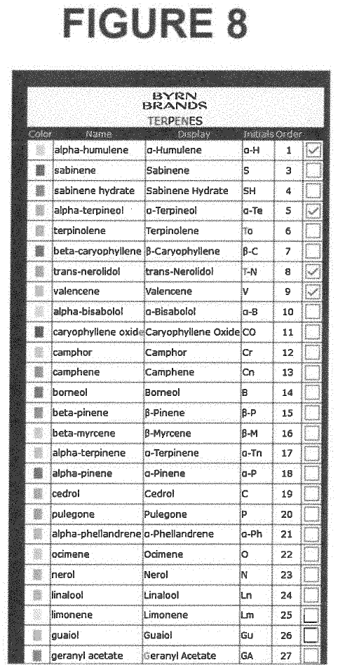

Turning to FIG. 8, when the “terpenes” button is selected, a list of available terpenes appears. In this embodiment, a user can check preferred terpenes for the final blend product, or in other embodiments, the system 10 can do this or other steps automatically. In some embodiments, such as those of FIGS. 7-8, the preferred terpenes and cannabinoids and amount can be selected, and the system can select preferred strains and amounts of strains to deliver these results.

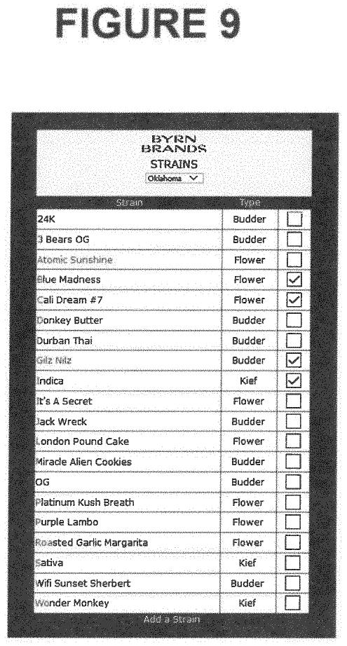

Turning to FIG. 9, strain, or selection of strains, can be chosen directly. In FIG. 9, a mix of strains is selected, and these strain(s) may already be pre-selected by the system 10. Alternatively, the system or user can also enter a set of preferred strains, along with cannabinoid and/or terpene preferences and amounts.

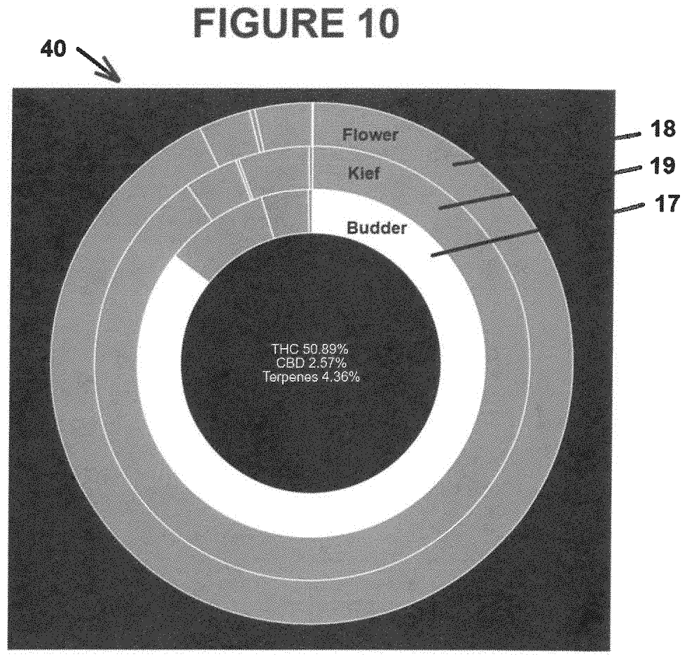

Turning to FIG. 10, a layered multi-product representation of cannabinoids in a profile blend product is shown. In this embodiment, there is a combination of kief 18, flower 18, and budder 17. As in previous visual representations, relative amounts of THC, CBD, and terpene compounds are shown in the center for reference. In this representation, a product of budder, flower, and kief is shown. However, in other embodiments, other mixes of types of product are possible, such as in earlier embodiments showing a combination of flower, oil, and kief.

Turning to FIG. 11, a complete visual representation of the blended strain product is shown. Terpenes are included, and as can be seen, this profile represents a mix of a multitude of compounds from various combined strains, showing some of the complexity the final blend, and system to create the blend, can incorporate.

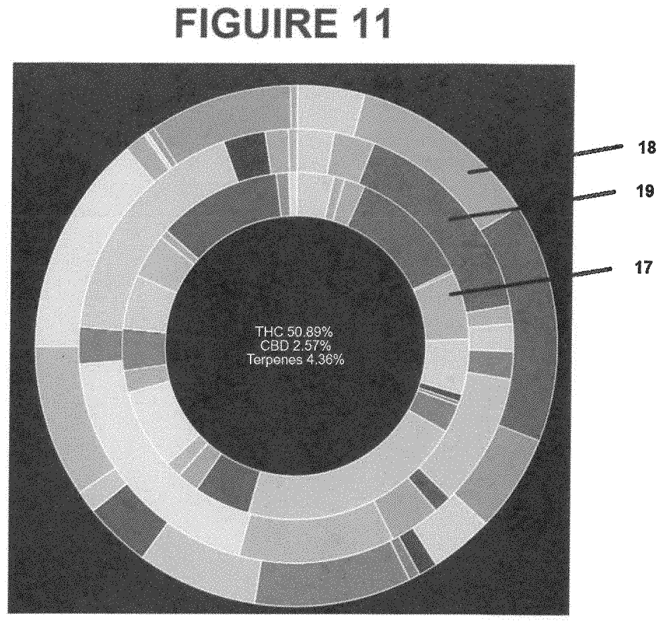

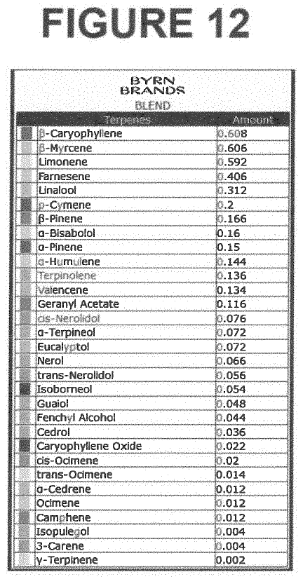

Turning to FIG. 12, the product of FIG. 11 is visually represented another way, with the compounds listed by percentages present.

One of the possible results of the system 10 and method herein is that a profile can be created with a high THC content, CBD content, or both. Cannabis products with high THC or CBD content are in much demand. These can be valuable in the treatment of anxiety, sleeping and eating disorders, or other mental health issues, arthritis, digestive issues, physical pain, symptoms, or other reasons. However, the physical process of creating a high-THC or high-CBD profile presents physical obstacles that currently make carrying it out difficult or impossible.

There is a physical limit to how much CBD or THC will be present naturally within a strain product. Accordingly, there is a natural limit on how much THC or CBD can be infused or placed within a cannabinoid product that is a blend of strain profiles, limiting its effectiveness. A regular Cannabis product is not able to achieve high or extremely high THC or CBD levels as well as some specific terpene and cannabinoid profiles herein.

Because of such limitations, some specific terpene and cannabinoid profiles, particularly those with high THC or CBD levels, cannot be utilized for purposes requiring high THC CBD content. Herein is a method and system for overcoming these difficulties via a “double dip” process to increase the infused amount of THC or CBD possible in a product.

In summation, these embodiment are for a process that involves blending and infusing a Cannabis product, which in some preferred embodiments, is a combination of Cannabis wet extracts, or wet and dry extracts, into a mash product, or other product, and then freeze drying the Cannabis mash product.

The freeze drying tends to pull or draw the wet extract component into the dry extract creating a more retentive product. When the combined product or fused product has been freeze dried, the product can then be infused with Cannabis wet extracts, wet and dry extracts, or dry extract, or a combination, again. This process raises the amount of Cannabis extracts that can be placed in the product. This increased dose can bring more effective pain relief to a user.

Turning to FIG. 13, a method is shown that is drawn to a double blending process 700 of the specific terpene and cannabinoid profiles created by the system 10 and method herein.

In a first step, the amount of THC and/or CBD percentage(s) desired are entered into the system 10 as parameters, using the electronic device 12 702. Other criteria, such as those previously noted for profile creation, such as, e.g., cost, profit, availability of strains, and inventory can also be included, or may be pre-entered into the system 10.

In a next step, the system 10 processes the entered (and possible pre-entered) parameters and delivers a set of profiles 704 that meet the high-THC, or high-CBD criteria.

Next, a specific profile of strain(s) and combined components, such as strain(s) of flower, kief, rosin, extracts, oils, hash, dry or wet Cannabis extract, or other product, or combination of these, is selected for a resultant profile of component(s) 706. Dry extracts can include, for a few examples, kief, thca, or hash. A few examples of wet extracts include bubble hash, shatter, crumble, budder, rosin, diamonds, sauce, distillate, or other Cannabis extracted oils such as butane hash oil, or oils derived from co2, ethanol, alcohol, and water or ice extraction methods. If desirable, the system 10 can be programmed to keep a detailed log of components and weight percentage of each component being used per batch, or an operator can keep a log manually.

In a further step, a first portion of the components are mixed and combined together 708. As steps toward this general step 708, any flower or coarse material is ground into a fine or medium coarse consistency. In an embodiment, a Cannabis grinder can be used to complete this. In one embodiment, the oil rosin, wet extract, or other liquid or partly-liquid substance is added and warmed, with a heated mixing machine.

In a next step, any rosin, oil, or wet extract, or other substance in liquid or partly-liquid form in the formulation is mixed with any ground flower in the formulation, along with any kief, thca or dry extract. As dry components such as kief/thca or dry extract are added, usually more fine components, then more coarse components are added as the mixture is stirred, typically into a mash.

In other embodiments, the mixed product, or mash, can be warmed, making use of a warming chain, or other temperature controlled process, primarily to convert any THCA into THC by heating, raising product effectiveness.

However, alternatively, a cold chain method, or any within the art, can be used to help preserve any vital terpenes and cannabinoids. Cold chain is a process in which specific or all profile strains are kept at a low temperature, or frozen, to preserve the chemical properties of the components within, particularly of the more fragile terpenes. The strain(s) can be kept at a temperature possibly as cold as—120 F or less.

In other embodiments, some components of a specific profile may be warmed while others are submitted to a cold chain process, and yet others may receive neither treatment, depending upon such factors as THC or CBD level sought, and terpenes to be present.

In a next step, the product which has been combined can be placed on trays and freeze-dried 710, which in an embodiment, can be done with a freeze dryer or other suitable apparatus in the art.

In a further step, a second portion of THC or CBD containing component or components, of the selected formulation, including components such as oil, rosin, kief, wet, or dry extract, or combination of these from the selected formulation are heated and combined 712, in one embodiment by placing the components into a heated mixing machine and mixing.

In another step, the first freeze dried portion is added to the heated and mixed second portion, and the two portions combined to form a THC-enhanced or CBD-enhanced, or both, formulation 714.

Finally, any further processing steps to prepare the product for sale or use can be completed 716. For example, in further manufacturing steps, the top of each product is secured, can be inspected, weighed, placed into approved packaging, and label stickers with pertinent information added to the packaging.

Turning also to FIGS. 14-16, an important aspect of the inventive method herein will be disclosed in further detail. Turning to FIG. 14, as well as FIG. 13, when the first portion of selected profile components are combined 708, they firm. a first portion of combined profile product 722. This first portion includes component or components high in THC, CBD or both 724. At least a portion of the high-THC/CBD components can be at the product surface 728, hut as shown by lines to several dots representing the high-THC/CBD components, the high-THC/CBD components are also spread generally throughout the combined profile product 722.

Turning to FIG. 15 as well as FIG. 13, when the first portion of combined product is freeze dried 710, the high-THC/CBD components tend to pull away from the surface 728 and as indicated by arrow set A, and saturate inward toward the center through absorption. This has the effect of physically leaving less high-THC/CBD product 724 at or near the product surface 728.

Turning to FIG. 16 as well as FIG. 13, when a second portion of combined components 726 are added to the freeze-dried first portion 712, because the high-THC/CBD components have been pulled toward the center, the combined profile product 722 is now able to physically accommodate high-THC/CBD product at and around the surface 728 yet again. The second high-THC/CBD portion 726 can then be combined and absorbed at the surface 728, increasing the amount of high-THC/CBD components within the combined profile product 722. By this method, the level of THC and CBD that can be obtained is increased over standard means of strain combination and mixing.

When the first and second portions are combined 714 and taken through any further processing steps 716, it will typically be part of a mass process in which multiple products are being manufactured through the same process, as shown by representative additional products 722a and 722b.

Disclosed herein is a method for increasing THC/CBD content in a cannabis product, thereby improving the range of available Cannabis products and available treatments.

It is to be understood that while certain forms of the present invention have been illustrated and described herein, the expression of these individual embodiments is for illustrative purposes and should not be seen as a limitation upon the scope of the invention. It is to be further understood that the invention is not to be limited to the specific forms or arrangements of parts described and shown.

1. A method for increasing THC or CBD content, or both, in a cannabis product, comprising the steps of
entering an amount of THC, CBD, or both into a cannabis calibration system as at least one parameter, using at least one electronic device,
processing the entered parameters and delivering at least one profile that meets the entered criteria,
selecting a profile comprised of at least two components, wherein at least one component is a wet component,
combining the at least two components into a combined cannabis product,
freeze-drying the combined cannabis product,
adding a second portion of at least one component containing THC car CBD, or both, to the combined cannabis product to form a THC-enhanced, CBD-enhanced, or both, product.

2. A method for increasing THC or CBD content, or both, in a cannabis product, according to claim 1, wherein the at least two components are comprised of
at least one dry component
and at least wet component.

3. A method for increasing THC or CBD content, or both, in a cannabis product according to claim 2, wherein the at least one dry component is flower, a dry extract, kief, thca, hash, or a combination of these.

4. A method for increasing THC or CBD content, or both, in a cannabis product according to claim 2, wherein the at least one wet component is
an oil, rosin, wet extract, or other liquid or partly-liquid substance, or combination of these.

5. A method for increasing THC or CBD content, or both, in a cannabis product according to claim 2, wherein the at least one wet component is
a wet extract, bubble hash, shatter, crumble, budder, rosin, diamonds, sauce, distillate, or other cannabis extracted oil, butane hash oil, an oil derived from co2, ethanol, alcohol, or water or ice extraction methods, or a combination.

6. A method for increasing THC or CBD content, or both, in a cannabis product according to claim 2, wherein the at least one dry component and the at least one wet component are combined into a mash.

7. A method for increasing THC or CBD content, or both, in a cannabis product according to claim 2, comprising the steps of
grinding any flower or coarse material into a fine or medium coarse consistency before combining the at least one dry component with the at least one wet component.

8. A method for increasing THC or CBD content, or both, in a cannabis product according to claim 2, comprising the steps of
warming at least a portion of the at least one wet component before combining it with the at least one dry component.

9. A method for increasing THC or CBD content, or both, in a cannabis product according to claim 8, wherein the at least a portion of the at least one wet component is warmed with a heated mixing machine.

10. A method for increasing THC or CBD content, or both, in a cannabis product, according to claim 1, comprising the further step of
entering cost, profit, availability of strains, inventory, flavor, or scent, or a combination, into the cannabis calibration system.

11. A method for increasing THC CBD content, or both, in a cannabis product according to claim 1, comprising the further step of
programming the cannabis calibration system to keep a log of components and weight/percentage of each component used per batch.

12. A method for increasing THC or CBD content, or both, in a cannabis product according to claim 1, comprising the further steps of
inspecting and weighing the product.

13. A method for increasing THC or CBD content, or both, in a cannabis product according to claim 12, comprising the further steps of
placing the product into packaging, and labeling.

14. A method for increasing THC or CBD content, or both, in a cannabis product according to claim 1, comprising the further steps of
warming at least one component of the combined cannabis product.

15. A method for increasing THC or CBD content, or both, in a cannabis product according to claim 1, comprising the further steps of
cooling at least one component of the combined cannabis product using a cold chain process.

16. A method for increasing THC or CBD content, or both, in a cannabis product according to claim 1, comprising the further steps of
warming at least one component of the combined cannabis product using a temperature controlled blending process,
cooling at least one component of the combined cannabis product using a cold chain process,
or both.

17. A method for increasing THC or CBD content, or both, in a cannabis product, comprising the steps of
selecting a cannabis profile derived from a cannabis calibration system,
wherein the cannabis profile is comprised of at least two components,
combining the at least one dry component with the at least one wet component into a combined cannabis product,
freeze-drying the combined cannabis product,
and adding a second portion of at least one component containing THC or CBD, or both, to the combined cannabis product to form a THC enhanced, CBD-enhanced, or both, product. The freeze drying process can be repeated.

18. A method for increasing THC or CBD content, or both, in a cannabis product according to claim 17, wherein
at least one component is at least one dry component
and at least one component is at least one wet component.

19. A method for increasing THC or CBD content, or both, in a cannabis product, comprising the steps of
blending a combination of cannabis wet extracts, or wet and dry extracts, into a mash product,
freeze drying the cannabis mash product,
and adding a second portion of at least one component containing THC or CBD, or both, onto the mash product. This freeze drying process can be repeated until desired THC or CBD content is attained.
